(19) United States
(12) Patent Application Publication
(10) Pub. No.: US 2010/0234945 A1
(43) Pub. Date: Sep. 16, 2010

(54) SURGICAL PROSTHESIS FOR PLASTIC RECONSTRUCTION

(76) Inventor: Patrick O'Leary, Dieburg (DE)

Correspondence Address: SUGHRUE MION, PLLC, 2100 PENNSYLVANIA AVENUE, N.W., SUITE 800, WASHINGTON, DC, 20037, US

(21) Appl. No.: 12/740,076

(22) PCT Filed: Oct. 28, 2008
(86) PCT No.: PCT/EP08/64604

§ 371 Date: Apr. 27, 2010

(30) Foreign Application Priority Data
Oct. 29, 2007 (FR) 07/58663

(51) Int. Cl. A61F 2/12

(52) U.S. Cl. 623/8

(57) ABSTRACT

A surgical prosthesis for implanting in a body for plastic reconstruction, said prosthesis being in contact with body tissue, the prosthesis being characterized in that it includes an outside surface that is hydrophilic so as to prevent at least in part the fixing and/or development of bacteria on said outside surface, in particular bacteria of the hydrophobic type.

The present invention relates to a prosthesis for implanting in a human body for plastic reconstruction, and more particularly a soft tissue body prosthesis, and more specifically a mammary prosthesis or implant.

A well-known problem that arises with mammary implants is capsular contracture. The formation of a capsule around a foreign object placed in the body is a normal consequence of implantation. Capsular contracture is the condition that occurs in about 5% of women in which the capsule surrounding the implant contracts in an attempt to isolate the implant from the remainder of the body because a bacterial infection has developed. If such capsular contracture is not treated, it can lead to extrusion of or definitive damage to the implant. Silicone elastomers have been used for a long time for fabricating devices suitable for being implanted in the human body because they are chemically stable with the body and non-toxic. Nevertheless, in spite of the relatively inert nature of silicone elastomers, they can give rise to a foreign-body reaction in certain patients. When a foreign substance penetrates into a human tissue, the natural and immediate reaction of the tissue surrounding the foreign substance is to make it inoffensive for the remainder of the body. A foreign body that is inert and voluminous is thus encapsulated in a layer of fibrous tissue in order to isolate it from the surrounding tissue. That is a defense mechanism that occurs in a process that is similar to forming scar tissue after an injury or a surgical incision. A capsule of fibrous tissue forms around and completely surrounds an implanted device, such as a permanent prosthesis, and this takes place in intimate manner, matching the respective shapes and curves of the implanted device.

Numerous hypotheses have been put forward to explain the mechanism of capsular contraction. Nevertheless, the most probable mechanism involves subclinical infection where bacteria that are normally present to some extent on the skin are introduced to the surface of the silicone implant while it is being put into place. These bacteria grow slowly and produce a biofilm on the surface of the prosthesis that is in contact with the surrounding tissues. The organisms constituting the biofilm may release exotoxins that may have a broad spectrum effect both locally and systemically. One of these effects is irritation of the capsule. It is assumed that when the capsule is irritated, the immune response of the body tends to isolate the implant in order to solve the problem, and that can result in capsular contracture.

The bacteria responsible for capsular contracture are generally slow-growing bacteria that are very hydrophobic in order to be capable of withstanding drying out on the skin. An example of such skin bacteria that are often isolated in association with capsular contracture is Staphylococcus epidermitis. That bacterium is opportunist and it is not normally associated with an infection. Nevertheless it is capable of producing a biofilm that surrounds the bacterium and that may protect it from the environment. The bacterium fixes preferentially to a solid support on which it can multiply. The layer of biofilm is the virulence factor that enables Staphylococcus epidermitis to survive where other bacteria would be destroyed by the normal immune system. The bacterium can thus live for long periods of time with a low metabolic rate.

It is practically impossible to sterilize skin completely. It is possible to reduce the number of organisms, but complete eradication is not possible. Consequently, there will always be bacteria on the skin that might contaminate the surgical field. The Staphylococcus epidermitis bacterium can thus become fixed on the silicone implant, in particular while it is being implanted, with a relatively large implant being passed through a relatively small incision in the skin. The implant becomes colonized by the bacterium because of the highly hydrophobic nature of the outside surfaces both of the implant (generally made of silicone for soft tissue prostheses) and of the bacterium, so they attract solids in non-specific manner. This enables a slow-growing biofilm to be formed on the outside surface of the implant. Because these bacteria grow slowly, they can adhere to the outside surface of the implant and remain dormant thereon over long periods of time in equilibrium with the host, sometimes for several years, and subsequently, e.g. because of stress on the body, the bacteria cease to live in equilibrium with the immune system of the body. At that moment, under such conditions for triggering disequilibrium, the bacteria may reproduce rapidly or at least exude exotoxins into their environment and cause inflammation on and around the capsule. Among conventional triggering conditions, mention may be made of immune status being lost or diminished because of illness, medical treatment, etc., that enable the bacteria to multiply.

Documents EP-0 057 033 and FR-2 822 383 describe methods of modifying the surface of mammary implants. Those methods are complex and therefore expensive to implement.

An object of the present invention is to provide a prosthesis for implanting in a human body for plastic reconstruction, which implant does not reproduce the above-mentioned drawbacks.

More particularly, an object of the present invention is to provide a prosthesis, in particular a silicone mammary prosthesis, for which the risks of capsular contracture are reduced.

Another object of the present invention is to provide such a prosthesis that is simple and inexpensive to fabricate and to make, and that does not require modifications to the implantation procedure performed by the surgeon.

The present invention thus provides a surgical prosthesis for implanting in a human body, as described in claim 1.

Advantageous embodiments are described in the dependent claims.

These characteristics and advantages and others of the present invention appear more clearly from the following detailed description of various embodiments, made with reference to the accompanying drawing, and given as non-limiting examples, in which:

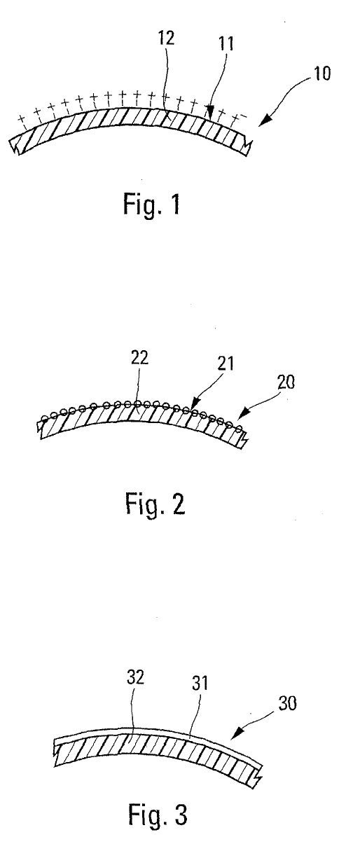

FIG. 1 is a cross-section view of a portion of a prosthesis, where the outside surface of the prosthesis is made of a silicone elastomer that has been chemically modified to make it hydrophilic;

FIG. 2 is a cross-section view of a portion of a prosthesis having a plurality of hydrophilic microparticles partially embedded in the outside surface of the prosthesis; and

FIG. 3 is a cross-section view of a portion of a prosthesis having a hydrophilic layer coated on the outside surface of the prosthesis.

The present invention consists in responding to the problem of post-implantation capsular contracture by attacking the hydrophobic/hydrophobic interaction between the surface of the prosthesis, which in the prior art is generally a hydrophobic surface, and potential colonizing bacteria, that are likewise hydrophobic. If the capacity of the outer surface for becoming a substrate or a medium for bacterial growth is disturbed, then the bacteria cannot attach to said surface and cannot produce a protective biofilm, with the resulting harmful consequences as described above. If the bacteria remain in hydrophilic tissue and/or in hydrophilic fluid, then they cannot become fixed to the surface of the prosthesis, and the normal immune response of the host will “clean out” the bacteria in known manner. The invention thus makes provision for causing the prosthesis, which is made of elastomer, in particular of silicone, to be hydrophilic so as to prevent any bacterial growth.

The preferred embodiment of the invention, which provides for chemically modifying the lateral and/or terminal groups of the silicone chain to make them more hydrophilic, is described in greater detail below. Preferably, the basic building blocks (or chains) of silicone, which are normally hydrophobic, are chemically modified to make the molecule hydrophilic. This may be done by substituting the methyl groups along the silicone backbone by any other group that is hydrophilic. Examples of such hydrophilic groups may be (in non-limiting manner): alcohols, ionic groups, organic molecules, etc., used singly or in combination with one another. Substitution may be performed at the end of the molecule, or along the length of the molecule, or as a combination of those two variants. The concentration of the substitution may be modified to vary the magnitude of the hydrophilic effect. Depending on the group that is used for substitution, and depending on concentration, silicone of hydrophilic nature may be restricted to the surface or may extend throughout the layer of silicone.

FIG. 1 is a cross-section view of a portion of a prosthesis 10 in which at least the outside surface 12 of the prosthesis is made of a silicone elastomer that is chemically modified 11 to cause the outside surface to be hydrophilic. In this preferred embodiment of the invention, the silicone is modified ab initio to make it hydrophilic. After curing, it is thus possible to form an implant that is hydrophilic from the start. In contrast, modifying the hydrophilic surface of an already existing implant, as described in documents EP-0 057 033 and FR-2 822 383 requires the use of means that are sophisticated and expensive. The present invention enables the desired hydrophilic properties to be obtained in a manner that is simpler and less expensive.

The entire prosthesis may be fabricated from the beginning using a hydrophilic silicone (or some other suitable elastomer). In a variant, the invention may also provide for making a prosthesis blank out of hydrophobic silicone and then dipping the blank one or more times in a modified silicone that is hydrophilic, so as to form one or more outer layers that are hydrophilic. After baking or curing, the resulting prosthesis presents an internal support structure that is hydrophobic, with an outside surface that is hydrophilic. Here likewise, there is no treatment of the outside surface of an implant previously made out of hydrophobic silicone, but on the contrary the outside surface of the prosthesis is made directly out of a hydrophilic material during the process of fabricating the prosthesis.

Several variants that may be used in addition are described below. These variants may include surface treatments of the implant in order to further improve the hydrophilic properties of said surface. They may also comprise using a bactericidal and/or bacteriostatic material, in particular silver, especially in ionic form, thereby having a non-toxic antimicrobial effect and thus being suitable for use in killing or eliminating bacteria should they nevertheless manage to develop on the surface of the prosthesis. The antimicrobial material may be embedded or contained in or on the surface of the prosthesis, and it may naturally be used together with hydrophilic surface properties for the prosthesis, as mentioned above.

Below, several additional embodiment variants are described in greater detail.

### 1. Chemical Modification of the Outside Surface of a Prosthesis

Although the outside surface of a prosthesis may comprise a wide variety of biocompatible elastomers, silicone is the elastomer that is most widely used for implantable soft tissue prostheses. Consequently, modifying the outside surface of a silicone prosthesis is described as a non-limiting example. The outside surface of a silicone implant of the invention may be made even more hydrophilic by covalently attaching thereto a hydrophilic end group, in particular at the end of the fabrication process, by putting the outside surface into contact with a reagent, such as cyanogen bromide. These types of agent activate the molecule to bind it covalently with other molecules, thereby fixing molecules preferentially on a zone in which they previously did not exist. Hydrophilic molecules may thus be fixed covalently in this way to the surface of the implant, making it even more hydrophilic.

In a variant, a hydrophobic end group may be fixed covalently to the silicone surface, and then subsequently separated, either chemically or biologically, so as to make the surface hydrophilic. Yet another method of making the outside surface of a silicone implant hydrophilic consists in coating the silicone outside surface of the implant with silicone comprising monomers including both a hydrophobic end group and an opposite hydrophilic end group so as to form the outermost layer. Such “bipolar” silicone may be fixed in hydrophobic manner to the silicone outside surface of the implant, while leaving the opposite hydrophilic group exposed to the environment. In a variant, the surface of the silicone implant may be coated with a hydrophobic-hydrophilic micelle.

### 2. Hydrophilic Particulate Coating

FIG. 2 is a cross-section view of a portion of a prosthesis 20 having a plurality of biocompatible hydrophilic particles 21 partially embedded in the outside surface 22. Such a prosthesis may be made by several fabrication methods. A preferred method comprises the steps of forming a silicone shell by repeatedly dipping a mandrel in the shape of a prosthesis in a silicone dispersion, with the coating thus formed between dips being cured. After the final silicone elastomer layer has been formed on the outside surface and the shell has the desired thickness, a coating of solid microparticles of a biocompatible hydrophilic material is applied to the surface before the outermost layer has been cured completely. The biocompatible hydrophilic material may be hydrophilic throughout the entire microparticle, or only at the surface of said microparticle. After the microparticles have been applied to the non-cured surface, the silicone is cured completely (e.g. by being heated), such that the microparticles have embedded portions embedded in the cured outer layer of silicone and exposed portions that extend outwards away from the silicone surface. The prosthesis as formed in this way may be implanted in a person's body.

3. Coating a Silicone Implant with a Biocompatible Hydrophilic Material

It is known that certain hydrophilic biopolymers, such as collagen and hyaluronic acid, and certain synthetic hydrophilic polymers such as polyvinyl pyrrolidone, having molecular weight of less than 50,000 AMU (atomic mass units), are tolerated by the body, metabolized, and/or excreted. Consequently, a silicone implant enveloped in a film comprising such polymers will present an outside surface that is hydrophilic. FIG. 3 is a cross-section view of a portion of a prosthesis 30 having a hydrophilic layer or film 31 coated on the outside surface 32 of the implant. Such a biocompatible hydrophilic film 31 may be fixed either to a smooth surface, or to a textured surface of a silicone implant.

4. The use of Hydrophilic Compounds that are Bonded in Non-Covalent Manner around the Implant

Any solution, cream, compound, material, or gel that is resorbable in the body and that is capable of interfering with the capacity for hydrophobic-hydrophobic interaction may be used at the time of surgery for modifying, breaking, or interrupting hydrophobic-hydrophobic interaction. By way of example, this may comprise (in non-limiting manner), lubricating materials for inserting the implant, anti-adhesion materials, betadine, etc. Such materials may be applied directly to the pocket for the implant, to the surface of the implant itself, or to a combination of both.

5. The use of Silver or of Compounds or of Nanoparticles Containing Silver to Interfere with Bacterial Growth in or on an Implant

Silver is well known for being a metal having therapeutic capacities with low toxicity for man. It may be bactericidal and/or bacteriostatic. Silver, or compounds containing silver, or nanoparticles of silver, in particular in ionic form, can be incorporated in the silicone or embedded in the surface of the prosthesis. Silver in any form whatsoever may be used in conjunction with an above-mentioned hydrophilic outside surface, or on its own.

Although the present invention is described with reference to a plurality of variants, it should be understood that a person skilled in the art may apply any useful modification thereto without going beyond the ambit of the present invention as defined by the accompanying claims.

1. A surgical prosthesis for implanting in a body for plastic reconstruction, said prosthesis being in contact with body tissue, the prosthesis being characterized in that it includes an outside surface that is hydrophilic so as to prevent at least in part the fixing and/or development of bacteria on said outside surface, in particular bacteria of the hydrophobic type.

2. A prosthesis according to claim 1, wherein said prosthesis is made of elastomer, in particular of silicone, said elastomer being modified and made hydrophilic prior to fabrication of said prosthesis.

3. A prosthesis according to claim 2, wherein said outside surface further includes a plurality of biocompatible hydrophilic microparticles partially embedded in the hydrophilic elastomer.

4. A prosthesis according to claim 2, wherein said outside surface is made even more hydrophilic by modifying silicone contained in said outside surface.

5. A prosthesis according to claim 2, wherein hydrophilic end groups are attached to the silicone by covalent bonding.

6. A prosthesis according to claim 2, wherein said outside surface includes a coating of a biocompatible hydrophilic film.

7. A prosthesis according to claim 2, wherein said outside surface is coated in a hydrophilic organic substance in covalent or ionic manner.

8. A prosthesis according to claim 1, wherein the outside surface of the prosthesis is coated by and/or contains a bactericidal and/or bacteriostatic material in order to eliminate and/or prevent growth and/or metabolization of bacterial contamination around, on, and/or in said prosthesis.

9. A prosthesis according to claim 8, wherein said bactericidal and/or bacteriostatic material comprises silver, silver compounds, or silver nanoparticles, or alloys including such a material.

10. A method of fabricating a prosthesis according to claim 1, the method comprising the steps of providing an elastomer, such as silicone, of modifying said elastomer to make it hydrophilic, and of using said modified elastomer for fabricating a prosthesis that is hydrophilic, at least in part.

11. A method according to claim 10, wherein a prosthesis blank made of hydrophobic elastomer is dipped, preferably several times, in a hydrophilic modified elastomer and is then cured, so as to fabricate a prosthesis comprising a hydrophilic internal support and a hydrophilic outside surface.

12. A method according to claim 10, wherein the step of modifying the elastomer is performed chemically by substituting the methyl groups of the elastomer molecules by hydrophilic groups.
